(19) United States
(12) Patent Application Publication
(10) Pub. No.: US 2015/0330983 A1
(43) Pub. Date: Nov. 19, 2015

(54) PEPTIDIC AND NON PEPTIDIC LIGANDS FOR IMMUNODETECTION OF THE RECEPTOR FOR UROTENSIN II

(71) Applicants: Ettore NOVELLINO, Montemarano (IT); Paolo GRIECO, Napoli (IT); Michele CARAGLIA, Ariano Irpino (IT); Alfredo BUDILLON, Napoli (IT); Renato FRANCO, Portici (IT); Santolo Rosaria ADDEO, Palma Campana (IT)

(72) Inventors: Ettore NOVELLINO, Montemarano (IT); Paolo GRIECO, Napoli (IT); Michele CARAGLIA, Ariano Irpino (IT); Alfredo BUDILLON, Napoli (IT); Renato FRANCO, Portici (IT); Santolo Rosaria ADDEO, Palma Campana (IT)

(73) Assignees: Ettore NOVELLINO, Montemarano (IT); Paolo GRIECO, Napoli (IT); Renato FRANCO, Portici (IT); Alfredo BUDILLON, Napoli (IT); Michele CARAGLIA, Ariano Irpino (IT)

(21) Appl. No.: 14/100,763

(22) Filed: Dec. 9, 2013

Related U.S. Application Data
(63) Continuation of application No. 12/449,513, filed on Aug. 10, 2009, now abandoned.

(30) Foreign Application Priority Data
Feb. 9, 2007 (IT) FI2007A000032

(51) Int. Cl. G01N 33/574

(52) U.S. Cl. CPC G01N 33/57434; G01N 2333/726

(57) ABSTRACT

The use of opportunely modified specific ligands of the receptor for urotensin II (UTR) or antibodies (commercially available) raised against the same receptor as tools for the definition of both the differentiation and the prognosis of human prostate adenocarcinoma is described, moreover, the use of opportunely radiolabeled ligands for UTR in the definition of the extension of disease is also described.

### FIELD OF THE INVENTION

The present invention is referred to cyclic peptides analogues of Urotensin-II of formula (I) modified through the covalent addition of an opportune marker, as biotin or other appropriate radioligands, in order to determine the diagnosis and prognosis of prostate adenocarcinoma and to their use in order to detect the prognosis of prostate adenocarcinoma.

### STATE OF THE ART

Urotensin-II is a cyclic peptide originally isolated from teleost fish urophysis and sequenced more than 20 years ago (Pearson, D. et al. Proc. Natl. Acad. Sci. USA (1980), 77, 5021-5024). Different structural forms of Urotensin-II were subsequently described in various species of fish and amphibians, as well as in mammals, including humans. In 1999 the Urotensin-II receptor was identified (Ames R. S. et al. Nature (1999), 401, 282-286). Several publications indicate that Urotensin-II is a potent constrictor of certain isolated human artery and veins, in vitro. Moreover, Urotensin-II induces contraction of non-vascular smooth muscle of cardiac and respiratory systems.

Urotensin-II and its receptor were identified in human cardiac tissues and it was reported that Urotensin-II exerts a strong inotropic effects in the human heart in vitro. Taken together, data reported in the literature indicate that Urotensin-II modulates cardiovascular homeostasis and therefore may play a key role in certain cardiovascular pathologies (Ames R. S. et al. Nature (1999), 401, 282-286). Consequently, synthetic analogues of Urotensin-II can be used to develop agonists, antagonists, and inhibitors of Urotensin-II and then for the pharmacological treatment of pathological states associated with Urotensin-II balance. For this purpose, cyclic peptides that are Urotensin-II analogues and comprise exclusively amino acid residues not containing sulphur, were developed and described in the International Patent Application No. WO 01/37856 (SmithKline Beecham Corp.).

Other cyclic peptides that are Urotensin-II analogues and have the above mentioned pharmacological use, were also described in the International Patent Application No. WO 01/37780 (SmithKline Beecham Corp.); in this case the peptides contain two cysteine residues linked by a disulphide bridge.

Cyclic peptide analogues of Urotensin-II having formula X-cyclo[M-A_{w}-B—C-D_{z}-N]-Y were described in Grieco P. et al., J. Med. Chem., 2002, 45:4391-4394; in these cyclic peptides a disulphide bridge is formed between the side chains of two amino acid residues containing sulphur M and N, which are never simultaneously cysteine; moreover, in the above said formula (I) w and z are 0 or 1, X is chosen among H, Ac, H-Asp, Ac-Asp, and H-Glu-Thr-Pro-Asp; Y is chosen among OH, NH₂, Val-OH, and Val-NH₂; A and D are chosen among Phe, D-Phe, Tyr, D-Tyr, Pro, D-Pro, Nal(1), D-Nal(1), Nal(2), D-Nal(2), Cha, Chg, e Bpa; B is chosen among Trp, D-Trp, e (α-Me)Trp; and C is chosen among Lys, Arg, Orn, Dap and Dab.

The above mentioned Urotensin-II peptide analogues described to date in the literature, although endowed with a certain antagonist activity toward Urotensin-II receptor, nevertheless show limited specificity and sometimes a residual agonist activity which severely limits their pharmacological and therapeutic usefulness. Therefore it is still felt the need of peptides endowed with antagonist activity toward the Urotensin-II receptor without the drawbacks reported above for the known peptides above reported, thus being useful for an effective treatment of the above mentioned pathological conditions.

Considering the high structural analogy between Urotensin-II and Somatostatin, the authors have performed bio-pharmacological studies to assess the involvement of this peptide-hormone in the expression and development of some important tumors, in particular the prostate carcinoma. Prostate adenocarcinoma is the fourth cause of death for tumours in Western Countries and represents one of the so-called “big killers”. Advanced and/or metastatic prostate cancer, in its hormone-refractory phase, is poorly responsive to conventional or alternative therapeutic approaches and the early onset of chemotherapy has demonstrated to give an improvement of the survival of this kind of patients. On the basis of these considerations, the definition of the clinical outcome of prostate cancer could be an important issue in the optimization of the therapeutic approaches. The definition of the clinical behaviour and the biological aggressiveness of this tumour is almost completely given by the study of the differentiation of the tumour based upon Gleason score. The latter is determined by the subjective examination of the tumour by pathologist who gives a morphologic score. High Gleason score is not always correlated to a worse prognosis and the finding of new and objective markers of differentiation in prostate cancer is mandatory for the definition of the clinical outcome. Another important issue in the characterization of metastatic prostate cancer is the determination of bone metastases that are not always clearly detectable with the conventional imaging procedures. The development of scintigraphy or PET-based procedures using radio-labelled UTR ligands could be useful in this specific setting in order to discriminate prostate cancer bone metastases.

### BRIEF DESCRIPTION OF THE FIGURES

FIG. 1 reports the evaluation of UTR on a panel of epithelial and haematological cancer cells.

FIG. 2 shows the immunohistochemical UTR evaluation in prostate adenocarcinoma

FIG. 3 shows the role of UTR in predicting the clinical outcome (survival) of prostate cancer patients independently from Gleason score.

FIG. 4 shows how the UTR expression allows the detection of patients with bad prognosis in Gleason >7 tumours

FIG. 5 shows how UTR expression predicts the clinical outcome of patients affected by T3-T4 tumours

### SUMMARY OF THE INVENTION

The application refers to cyclic peptides of general formula (I) containing a marker at N-terminal position:

Bio-X-ciclo[Z-A-B—C-D-Cys]-Y (I)

wherein:
X is selected from the group consisting of H, H-Asp, H-Phe, H-D-Phe, Gly, Gly-Gly, H-Glu-Thr-Pro-Asp, and other spacers.
Y is selected from the group consisting of OH, NH₂, Val-OH, Val-NH₂, Ile-OH and Ile-NH₂.
A and D, equal or different from each other, are selected from the group consisting of Phe, D-Phe, Tyr, D-Tyr, Cyclohexil-alanine and Cyclopentil-alanine.
B is selected from the group consisting of D-Trp, D-(α-Me)Trp, D-Trp(Me), D-Trp(CHO), D-Nal(1) and D-Nal(2);
C is selected from the group consisting of Lys, Asp, Glu, Orn, Cit, Dap, Dab and (p-amino)Phe;

### Z is Pen or Cys

Bio is a marker;
or pharmaceutically acceptable salts thereof.

### DETAILED DESCRIPTION OF THE INVENTION

The present invention makes available cyclic peptides, opportunely marked, capable of specifically binding to Urotensine II receptors that are present on the surface of cancer cell in the prostate adenocarcinome.

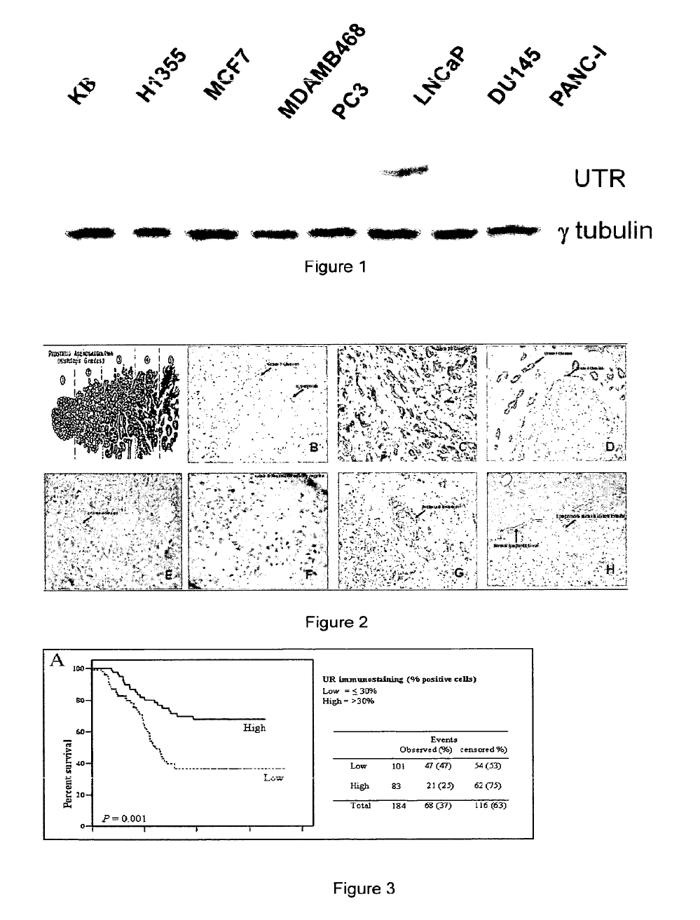

In fact it was now found that UTR protein is expressed at high levels in human androgen-dependent prostate adenocarcinoma LnCaP cell line after evaluation of UTR expression in a wide panel of human tumour cell lines. In details, UTR was evaluated in the three “classical” cell lines of human prostate carcinoma, PC3, LNCaP and DU145, by Western Blotting method. UTR is highly expressed in LNCaP cell line while its exporession is low or undetectable in the other two cells (FIG. 1).

On the basis of these results it was also surprisingly found that UTR is expressed at high levels in prostate adenocarcinoma samples collected from patients subjected to tumour biopsy and/or radical prostatectomy. The expression of UTR can be evaluated with immunohistochemical methods using an antibody already commercially available. In detail, UTR has been found to be specifically expressed in low Gleason grade prostate adenocarcinoma while its expression was gradually lost in high Gleason grade and completely undetectable in Gleason 5. The overall survival of the patients was moreover correlated to the expression of UTR that was also able to discriminate the patients with good prognosis from those with bad prognosis in the group of patients with Gleason Grade >7. UTR is in our series an independent prognostic marker and is strongly correlated to Gleason score and tumour stage. Therefore, UTR appears an objective marker that allows the optimization of surgical treatment and predicts the responsiveness to medical treatments of human prostate adenocarcinoma. We have also modified cyclic peptides with further reported formulas (I) and correlated to P5U and Urantide compounds through the covalent link of biotin and we have found that the latter are able to specifically bind the UTR and to detect its expression through the development of a colorimetric reaction. They can be therefore used as diagnostics in the molecular characterization of human prostate adenocarcinoma. Moreover, they can detect UTR expression in fresh tumour tissues collected from patients during the surgical procedures for extemporaneous evaluation.

Subject of the present invention are therefore cyclic peptides of general formula (I) marked at N-terminal position of this sequence:

Bio-X-ciclo[Z-A-B—C-D-Cys]Y (I)

wherein:
X is selected from the group consisting of H, H-Asp, H-Phe, H-D-Phe, Gly, Gly-Gly, H-Glu-Thr-Pro-Asp, and other spacers.
Y is selected from the group consisting of OH, NH₂, Val-OH, Val-NH₂, Ile-OH and Ile-NH₂.
A and D, equal or different from each other, are selected from the group consisting of Phe, D-Phe, Tyr, D-Tyr, Cyclohexil-alanine and Cyclopentil-alanine.
B is selected from the group consisting of D-Trp, D-(a-Me)Trp, D-Trp(Me), D-Trp(CHO), D-Nal(1) and D-Nal(2);
C is selected from the group consisting of Lys, Asp, Glu, Orn, Cit, Dap, Dab and (p-amino)Phe;

### Z is Pen or Cys;

Bio is a marker;
or pharmaceutically acceptable salts thereof.

According to the present invention marker is a Fluorofore agents or a radiomarker with radioactive isotope.

Examples of fluoroforic markers according to the invention are: biotin, fluoresceine isothiocianate, rodamine and similar marking compounds.

Radioactive isotope useful for the invention are for example those used for Imaging application as PET or SPECT, for example: ^{99m}Tc, ¹²⁴I, ^{110m}I, ¹⁸F, ⁶⁸Ga, ⁴⁴Sc, ⁸⁶Y, etc.

Further subjects of the invention are the pharmaceutical compositions comprising the above said cyclic peptides of formula (I) and/or their salts.

Further subject of the invention is the use of the cyclic peptides of formula (I) and/or of their salts as reagents for the pharmacological characterization of Urotensin-II receptors, and their use for the preparation of pharmaceutical compositions for the treatment of pathological conditions associated with unbalance of Urotensin-II.

The present peptides contain a disulphide bridge between Pen/Cys (position 5) and Cys (position 10) residues and are characterized by the presence of an amino acid residue B of the D series and by a suitable combination of amino acid residues B and C as reported above. Also, at N-terminal position all these peptides contain an appropriate marker, like as a fluorofore or radioligand agent.

The compounds described in this invention can be prepared by conventional solution and solid-phase peptide synthesis, known to any person skilled in the art. Thus, for example, to prepare peptides with a free acid at C-terminal, the synthesis in solid-phase can be performed using a Wang resin. Instead, to obtain peptides as C-terminal amide the synthesis in solid phase can be performed using a resin like as a PAL resin [Tris(alkoxy)benzylamide].

The synthesis of the peptides object of this application can be performed by using orthogonal protection using Fluorenylmethoxycarbonyl (hereinafter referred to as “Fmoc”) and t-Butyloxycarbonyl (hereinafter referred to as “Boc”) as protecting groups at a-amino group of amino acids, and t-Butyl (hereinafter referred to as “t-Bu) and Benzyl (hereinafter referred to as “Bzl”) as protecting groups at side-chain of amino acids, such as Asp, Glu (as t-butyl ester), Lys (Boc), Tyr, Thr (OtBu).

The thiolic group of Cys and Pen can be protected by a Trityl group (hereinafter referred to as “Trt”).

Regarding the strategy “Fmoc/t-Bu”, the peptide synthesis consists of the following steps:
a) loading of first C-terminal residue onto the resin;
b) deprotection of a-amino group by a solution of piperidine 25% (v/v) in dimethylformamide (DMF);
c) coupling of the next amino acid, appropriately protected as above said, using a condensing or activating agent, such as N,N′-dicyclohexylcarbodiimide (DCC), O-(benzotriazole-1-yl)-1,1,3,3-tetra-methyluronium hexafluorophosphate (HBTU), O-benzotriazole-1-yl-1,1,3,3-tetramethyluronium tetrafluoroborate (TBTU), 1-hydroxy-benzotriazole (HOBt), etc.;
d) repeating of steps a) and b) until the desired sequence is completed;
e) cleavage of the linear peptide from the resin and deprotection of amino acids side-chains by using trifluoroacetic acid in the presence of suitable “scavengers”, such as triethylsilane (TES) and water (5% v/v, each);
f) formation of disulfide bridge by oxidation, for example with Ferricyanide, oxygen or dimethyl sulfoxide (DMSO);
g) purification of crude cyclic peptide by semipreparative HPLC, preferably by reverse-phase HPLC on C-18 column, using as eluents acetonitrile and water in the presence of 0.1% of trifluoroacetic acid (TFA);
h) lyophilization of final purified product;
i) characterization of final compound by analytical HPLC, Mass spectrometry (MS) and amino acids analysis (AAA).

The present peptides of formula (I), in free form or as pharmaceutically acceptable salts, can be used for the preparation of pharmaceutical compositions according to conventional methods of preparation, well-known in pharmaceutical field.

These pharmaceutical compositions can be conventionally formulated, and may further comprise one or more pharmaceutically acceptable excipients and/or diluents.

The administration of the present compositions is feasible by any conventional way, for example by parenteral injection in the form of injectable solutions or suspensions, or by oral, topic, nasal administration, etc.

The formulations of the present peptides include compresses, capsules, pills, solutions, dispersions, creams and ointments, emulsions, aerosol, and can also be used to accomplish a controlled or delayed release of the active compound.

The present pharmaceutical compositions may comprise the peptides of formula (I) according to the invention as sole active component, or may comprise the present peptides in combination with other active compounds or adjuvants, suitably chosen according to the pathological conditions to be treated.

The pharmaceutical compositions comprising the present peptides are useful for the pharmacological treatment of pathologies associated with an alteration of Urotensin-II balance, such as hypertension, heart arrhythmia, heart stroke, angina, ischemia of myocardium and restenosis.

The present peptides of general formula (I) can also be used as reagents in biotechnological and pharmaceutical research, for example as reagents in characterisation of human Urotensin-II receptor.

According to the present invention the natural amino acids were indicated with the three letters codex commonly used; the amino acids which are not genetically codified were indicated as follows

### Pen Penicillamine;

### Nal(1) 3-(1-Naphtyl)alanine;

### Nal(2) 3-(2-Naphtyl)alanine;

### Orn Ornitine;

### Cit Citrulline;

Dap 2-amino-propionic acid.
Dab diammino-butiryc acid

### Cha Cyclohexyl-alanine

Tic Tetrahydroisoquinolinic-3-carbossilico
Ac is an acetyl group.

### Example 1

The Biotilinated compound shows the following structure:

Biotin-Asp-c[Cys-Phe-Trp-Lys-Tyr-Cys]-Val-NH₂

The compound was synthesised by standard solid-phase methodology, starting from 0.5 g of Wang resin (0.7 mmol/g) using N^{α}-Fmoc chemistry and an orthogonal side chain protection strategy. The first amino acid, N^{α}-Fmoc-Val-OH, was linked to the resin using as coupling reagents a 3-fold excess of HBTU/HOBt in the presence of N,N′-diisopropylethylamine (DIEA). The following amino acids were then added stepwise to the growing peptide: N^{α}-Fmoc-Cys(Trt)-OH, N^{α}-Fmoc-Tyr(OtBu)—OH, N^{α}-Fmoc-Orn(Nε-Boc)-OH, N^{α}-Fmoc-D-Trp(Nin-Boc)-OH, N^{α}-Fmoc-Phe-OH, N^{α}-Fmoc-Pen(Trt)-OH and N^{α}-Fmoc-Asp(OtBu)—OH using standard solid phase methods. The last amino acid, N^{α}-Fmoc-Asp(OtBu)—OH, after deprotection of protecting group Fmoc is coupled Biotin or other Fluoroforic agent by conventional coupling reagent.

Each coupling reaction was achieved using a 3-fold excess of amino acid and of HBTU/HOBt in presence of DIEA. The N^{α}-Fmoc protecting group was removed by treating the protected peptide resin with 25% piperidine solution in DMF (1×50 mL, 5 min, 1×50 mL, 20 min). The peptide resin was washed with DMF (3×50 mL), DCM (3×50 mL) and again with DMF.

Upon complete formation of the protected linear peptide, the compound was cleaved from the resin and the other side chain protecting groups removed using the following mixture: TFA/TES/H2O (9:0.5:0.5) for 3 h. All procedures were done under an Argon atmosphere. The resin was removed from solution by filtration and the crude peptide was recovered by precipitation with cold anhydrous ethyl ether giving a white powder that was purified by preparative HPLC on a C18-bonded silica gel column (Vydac 218TP1010, 1.0×25 cm) eluted with a linear gradient of acetonitrile in aqueous 0.1% of TFA (v/v). The purification was monitored at 280 nm, and the fraction corresponding to the major peak were collected, combined, and lyophilised to give the final compound as a pure (>98%) white powder. The linear peptide was characterised by analytical HPLC and FAB-MS (m/e 1073.282 [M+]). The final cyclic peptide, containing the disulfide bridge, was achieved by oxidation with an aqueous solution of potassium ferricyanide at pH=8.5 (ammonium buffer). This step was monitored by analytical HPLC to evaluate the grade of conversion. Final cyclic peptide was purified by reverse-phase HPLC using the condition above described.

### Example 2

### Labelling of Tumour Cells Collected with Fine Needle Aspiration Biopsy

Cells are centrifuged with Cytospin and subsequently fixed at the air for 30 min and then with the addition of a 1:1 acetone/methanol buffer for another min. The fixed cells are incubated in 1% H₂O₂/methanol for 20 min at room temperature in order to block endogeneous oxidases. Thereafter, cells are incubated in 10% PBS/BSA buffer for 10 min and with the biothynilated UTR ligand in the presence or absence of unlabelled UTR ligands for 90 min at room temperature in order to displace labelled ligand and to give an idea on the specificity of the reaction. Then cells are washed with 10% PBS/BSA buffer for 2 min and incubated with DAKO ABC complex per 40 min. After the incubation cells are washed in PBS/BSA buffer and Diaminobenzidin is added for 10 min. Thereafter, the cells are washed again in PBS/BSA for 10 min and are finally died with hematoxilin for 30 sec and dehydrated with alcool, diaphanyzed with xylene and mounted in Istomount.

### Example 3

### Labelling of Tumour Cells in Surgical Samples

Freeze tissue slides are obtained from fresh tissues collected form surgical samples (after either biopsies or radical prostathectomies). The slides are incubated in 1% H₂O₂/methanol for 20 min at room temperature in order to block endogeneous oxidases. Thereafter, slides are incubated in 10% PBS/BSA buffer for 10 min and with the biothynilated UTR ligand in the presence or absence of unlabelled UTR ligands for 90 min at room temperature in order to displace labelled ligand and to give an idea on the specificity of the reaction. Then slides are washed with 10% PBS/BSA buffer for 2 min and incubated with DAKO ABC complex per 40 min. After the incubation slides are again washed in PBS/BSA buffer and Diaminobenzidin is added for 10 min. Thereafter, slides are washed again in PBS/BSA for 10 min and are finally died with hematoxilin for 30 sec and dehydrated with alcool, diaphanyzed with xylene and mounted in Istomount.

### Example 4

### Immunohistochemical Detection of UTR in Surgical Samples

Tissue samples are fixed in 10% buffered neutral Formalin, included in paraffin and 5μ sections are obtained. All sections then were de-paraffinized in xylene (BioClear, three times for 15 min), rehydrated through a graded alcohol series (95% alcool to discard xylene, followed by 90% and 70% alcool for 30 min) and finally washed in PBS. This buffer was used for all subsequent washes and for dilution of the antibodies. The slide were incubated in Cytrate buffer (ph 6.0) in microwave owen three times for 5 min in order to unmask antigenic sites. Thereafter, the slides are incubated in 100 μl H₂O₂/methanol 0.5% v/v for 10 min at room temperature. After incubation the slides are washed twice with TBS/tween 20 buffer for 5 min. In this way the endogenous peroxidase is blocked. Then the slides are incubated in primary antibody (anti-UTR antibody purchased from Santa Cruz, Santa Cruz, Ca) at 1:3000 dilution for 1 h at room temperature. Then the slides are washed twice with TBS/tween 20 buffer for 5 min. The slides are incubated with a secondary goat anti-rabbit antibody linked through a dextrane polymer to horse radish peroxidase. After incubation for 30 min at room temperature the slides are washed twice with TBS/tween 20 buffer for 5 min. The sections are covered with 100 μl chromogen substrate (DAB, 3,3′-Diaminobenzidin and 2.5-3% H₂O₂) and incubated for 10 min at room temperature at dark. Thereafter, the slides are washed again in distilled water to discard Diaminobenzidin and counterstained with ematoxilin for about 1 min followed by washing in water for at least 5 min. The slide were dehydrated through a graded alcohol series, clarified in xylene and mounted for visualization at optical microscope (Zeiss).

The UTR expression has been evaluated on 200 in vivo samples collected from patients affected by prostate adenocarcinoma. UTR expression has been correlated with different clinical and pathological parameters including Gleason score, androgen receptor expression, age and TNM.

The expression was very high in Gleason 2-3 (C and D) and absent in Gleason 5 (F). UTR was expressed in prostate tumour cells infiltrating a nerve (G) and a node (H) (FIG. 2).

The correlation between UTR expression and low Gleason score was statistically significant (p=0.001) while it did not correlate with either age (p=0.4) or stage (p=0.007) (Table 1).

TABLE 1
UTR expression in relation to clinical and pathological
parameters in a series of 195 prostate cancer patients

| | | Low | High | |
|---|---|---|---|---|
| | Total | n (%) | n (%) | |
| | 195 | 106 (54) | 89 (46) | p value |
| Age (yr) | | | | |
| ≦70 | 109 | 62 (57) | 47 (43) | 0.4 |
| >70 | 86 | 44 (51) | 42 (49) | |
| Tumor stage* | | | | |
| pT2 | 67 | 29 (43) | 38 (57) | 0.07 |
| pT3 | 62 | 36 (58) | 26 (42) | |
| pT4 | 66 | 41 (62) | 25 (38) | |
| Gleason score* | | | | |
| Low | 50 | 16 (32) | 34 (68) | 0.001 |
| Medium | 70 | 34 (49) | 36 (52) | |
| High | 75 | 56 (75) | 19 (25) | |

The overall survival of patients having more than 30% of cells expressing UTR was significantly higher than those with low UTR expression (p=0.001) (FIG. 3) that was also an independent prognostic factor together with stage Table 2.

TABLE 2
Contribution of various potential prognostic factors to overall
survival by Cox regression analysis in prostate cancer patients

| | Risk | 95% confidence | |
|---|---|---|---|
| Variable | ratio | interval | p value |
| Age§ | 1.412 | 0.876-2.276 | 0.157 |
| Gleason score* | 1.012 | 0.547-1.907 | 0.948 |
| Tumor stage# | 3.296 | 1.712-6.347 | 0.001 |
| UR** | 2.250 | 1.323-3.828 | 0.003 |

§The risk ratio is given as older versus younger patients (cut-off = 70 years)
*The risk ratio is given as higher (>7) versus lower Gleason score.
#The risk ratio is given as higher (pT4) versus lower stage cancers.
**The risk ratio is given as low (≦30% positive cells) versus high (>30%) expressor tumors.

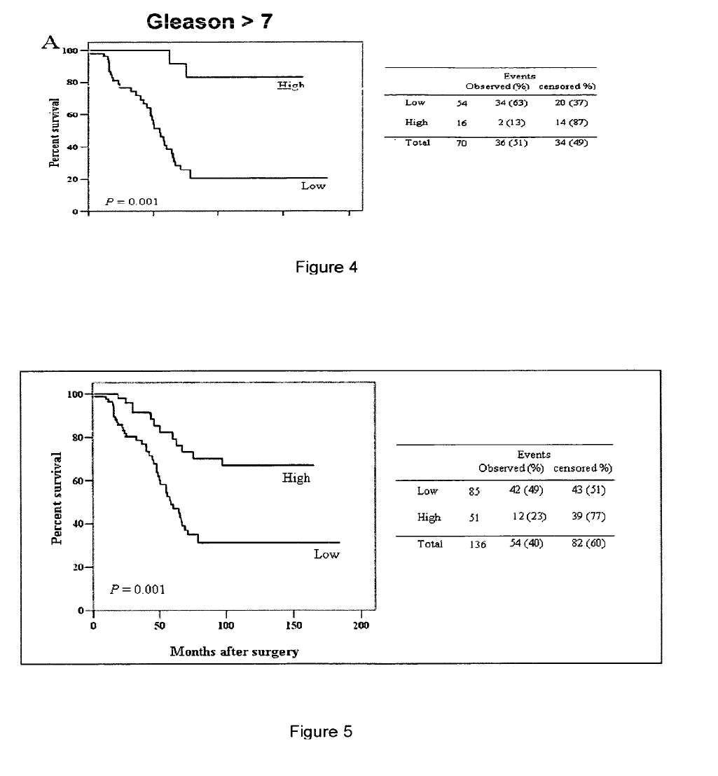

Interestingly, UTR was able to discriminate the sub-group of patients with good prognosis when Gleason score was more than 7 (p=0.001) (FIG. 4).

UTR preserved its discriminating function also in pT2-pT3 and pT3-pT4 patient subgroups (FIG. 5). On the other hand, the correlation between UTR and androgen receptor expression was not significant (p>0.05).

The measurement of UTR expression in prostate cancer is useful to establish the prognostic score of the patients per se and independently (being UTR and independent prognostic factor) from Gleason score that remains a subjective morphologic parameter of evaluation that can change from a pathologist to another. In fact, this kit is based on semi-quantitative parameters (more or less than 30% of cells positive for UTR expression) and, therefore, provides subjective criteria of evaluation of the grade of differentiation of the tumor.

The evaluation of UTR expression is also useful to discriminate the patients with good prognosis in the subset group with Gleason score more than 7. The definition of the prognosis of this subset of patients is an important and debated issue. In fact, part of these patients continues to have a long-lasting androgen-responsive disease while another group has a disease rapidly developing in aggressive and hormone-refractory cancer. Therefore, the early definition of the clinical outcome of these patients has important consequences on the choice of the optimal self-tailored therapeutic strategy. The availability of a cheap and easy maker assay that can drive the choice of the optimal therapy will have a rapid and wide diffusion in the clinical practice.

We have also performed binding assays on freezed tumour slides of cyclic biothinilated peptides and we have demonstrated a specific binding of the latter peptides to UTR. In fact, they can be displaced by saturatine concentrations of urotensin-II. These results confirm for the first time that these modified peptides can specifically bind only the UTR and therefore can be used as specific probes in the detection of the expression of the above entioned receptor in fresh prostate cancer tissues. Therefore, this method can be also used in the extemporaneous determination of UTR expression in order to give additional information to the surgeon about the intervention on the basis of biological aggressiveness of the neoplasm.

1.-24. (canceled)

25. A method for diagnosis of prostate adenocarcinoma wherein a cycled peptide having generic formula containing Biotine at N-terminal position:
Bio-X-ciclo[Z-A-B—C-D-Cys]Y (I)
wherein:
X is selected from the group consisting of H, H-Asp, H-Phe, H-D-Phe, Gly, Gly-Gly, H-Glu-Thr-Pro-Asp, and other spacers;
Y is selected from the group consisting of OH, NH2, Val-OH, Val-NH2, Ile-OH and Ile-NH2;
A and D, equal or different from each other, are selected from the group consisting of Phe, D-Phe, Tyr, D-Tyr, Cyclohexil-alanine and Cyclopentil-alanine;
B is selected from the group consisting of D-Trp, D-(a-Me)Trp, D-Trp(Me), D-Trp(CHO), D-Nal(1) and D-Nal(2);
C is selected from the group consisting of Lys, Asp, Glu, Orn, Cit, Dap, Dab and (p-amino)Phe;
Z is Pen or Cys;
Bio is a marker
is used for binding with UTR II receptors in a sample of a human prostatic tissue and evaluating the UTR expression for characterizing the prostate adenocarcinoma.

26. The method according to claim 25, wherein the UTR expression is detected in a surgical sample evaluated with an immunoistochemical method.

27. The method according to claim 26, wherein an indirect immunhoistochemical method with a primary and a secondary antibodies is used.

28. The method according to claim 25, wherein the UTR expression is detected on a frozen tissue.

29. The method according to claim 25, wherein the UTR expression is determined independently from the Gleason grade.

30. The method according to claim 25, wherein adenocarcinoma is characterized for patients having a Gleason grade higher than 7.

31. The method according to claim 25, wherein adenocarcinoma is characterized for patient subgroups with stage II-III or III-IV.


SEQUENCE LISTING (11 sequences)

SEQ ID NO: 1 - protein, 8 aa, Artificial
  synthetic peptide
DXFWKYCV

SEQ ID NO: 2 - protein, 8 aa, Artificial
  Synthetic peptide
DCFWKYCV

SEQ ID NO: 3 - protein, 8 aa, Artificial
  Synthetic peptide
DXFWXYCV

SEQ ID NO: 4 - protein, 8 aa, Artificial
  Synthetic peptide
DXFWKYCV

SEQ ID NO: 5 - protein, 8 aa, Artificial
  Synthetic peptide
DXFWXYCV

SEQ ID NO: 6 - protein, 11 aa, Artificial
  Synthetic peptide
ETPDXFWKYCV

SEQ ID NO: 7 - protein, 11 aa, Artificial
  Synthetic peptide
ETPDXFWXYCV

SEQ ID NO: 8 - protein, 8 aa, Artificial
  Synthetic peptide
DXFWXYCV

SEQ ID NO: 9 - protein, 8 aa, Artificial
  Synthetic peptide
DXFWXYCV

SEQ ID NO: 10 - protein, 7 aa, Artificial
  Synthetic peptide
XFWKYCV

SEQ ID NO: 11 - protein, 7 aa, Artificial
  Synthetic peptide
XFWXYCV